(19) United States
(12) Patent Application Publication
(10) Pub. No.: US 2019/0085208 A1
(43) Pub. Date: Mar. 21, 2019

(54) POLISHING COMPOSITION, METHOD FOR PRODUCING SAME, POLISHING METHOD, AND METHOD FOR PRODUCING SUBSTRATE

(71) Applicant: FUJIMI INCORPORATED, Kiyosu-shi (JP)

(72) Inventors: Toshio SHINODA, Aichi (JP); Aya NISHIMURA, Aichi (JP)

(73) Assignee: FUJIMI INCORPORATED, Kiyosu-shi (JP)

(21) Appl. No.: 16/129,665

(22) Filed: Sep. 12, 2018

(30) Foreign Application Priority Data
Sep. 15, 2017 (JP) 2017-177989

(51) Int. Cl. C09G 1/02; C07D 257/06

(52) U.S. Cl. CPC C09G 1/02; H01L 21/30625; C07D 257/06

(57) ABSTRACT

To provide a polishing composition capable of polishing objects to be polished, such as simple substance silicon, silicon compounds, and metals, at a high polishing removal rate.

The polishing composition contains a polishing removal accelerator containing a compound having a ring structure configured to contain four or more nitrogen atoms, abrasives, and a liquid medium.

### BACKGROUND OF THE INVENTION

### Field of the Invention

The present invention relates to a polishing composition, a method for producing the same, a polishing method, and a method for producing a substrate.

### Description of the Related Art

A process for manufacturing a semiconductor device includes a process of polishing objects to be polished, such as simple substance silicon (Si), silicon compounds, and metals, in which it is required to polish silicon nitride (Si₃N₄) having poor chemical reactivity, for example, at a high polishing removal rate. Most of polishing compositions used heretofore for polishing the silicon nitride contain abrasives and acids. For example, PTL 1 discloses a polishing composition containing phosphoric acid or a phosphoric acid derivative. Moreover, PTL 2 discloses a polishing composition containing colloidal silica and organic acid having a sulfonic acid group or a phosphonic acid group. Furthermore, PTL 3 discloses an acidic polishing composition containing colloidal silica in which organic acid, such as sulfonic acid or carboxylic acid, is immobilized. However, these conventional polishing compositions have not sufficiently satisfied a demand for the polishing removal rate of the silicon nitride of a user.

### CITATION LIST

### Patent Literatures

PTL 1: JP 6-124932 A

PTL 2: JP 2010-41037 A

PTL 3: JP 2012-40671 A

### SUMMARY OF THE INVENTION

Then, it is an object of the present invention to solve the problems of the conventional techniques described above to provide a polishing composition capable of polishing objects to be polished, such as simple substance silicon, silicon compounds, and metals, particularly silicon nitride, at a high polishing removal rate, a method for producing the same, a polishing method, and a method for producing a substrate.

In order to solve the above-described problems, a polishing composition according to one aspect of the present invention contains a polishing removal accelerator containing a compound having a ring structure configured to contain four or more nitrogen atoms, abrasives, and a liquid medium.

In the polishing composition according to the above-described one aspect, the number of ring members of the ring structure may be 5 or more and 14 or less. The compound having the ring structure may be one containing at least one group selected from the group consisting of an amino group, an amide group, a phenyl group, a carboxy group, a phosphate group, a sulfo group, and a thiol group. The compound having the ring structure may be at least one type selected from the group consisting of tetrazole and a derivative thereof. The tetrazole and the derivative thereof may be at least one type selected from the group consisting of 1H-tetrazole, 5-amino-1H-tetrazole, 5-phenyl-1H-tetrazole, and 5,5′-bistetrazole diamrnonium.

Furthermore, in the polishing composition according to the above-described one aspect, the concentration of the compound having the ring structure may be 1 mmol/L or more and 100 mmol/L or less.

Furthermore, the polishing composition according to the above-described one aspect can be used for polishing of a substrate having a positively charged region in a state where the pH is 6 or less.

Furthermore, a method for producing a polishing composition according to another aspect of the present invention is a method for producing the polishing composition according to the above-described one aspect and has a process of mixing the polishing removal accelerator, the abrasives, and the liquid medium.

Moreover, a polishing method according to another aspect of the present invention has a process of polishing a polishing object to be polished using the polishing composition according to the above-described one aspect. In the polishing method, the object to be polished may be silicon nitride.

Furthermore, a method for producing a substrate according to another aspect of the present invention has a process of polishing the surface of a substrate using the polishing composition according to the above-described one aspect.

The polishing composition and the polishing method of the present invention enable polishing of objects to be polished, such as simple substance silicon, silicon compounds, and metals, at a high polishing removal rate. Moreover, the method for producing a polishing composition of the present invention can produce a polishing composition polishing objects to be polished, such as simple substance silicon, silicon compounds, and metals, at a high polishing removal rate. Moreover, the method for producing a substrate of the present invention can produce a substrate which contains objects to be polished, such as simple substance silicon, silicon compounds, and metals, and the surface of which is polished.

### BRIEF DESCRIPTION OF THE DRAWINGS

FIG. 1A is a side view schematically illustrating a state where 1H-tetrazole approaches an object to be polished.

FIG. 1B is a side view schematically illustrating a state where 5-phenyl-1H-tetrazole approaches an object to be polished.

### DESCRIPTION OF THE PREFERRED EMBODIMENTS

An embodiment of the present invention is described in detail. A polishing composition of this embodiment contains a polishing removal accelerator containing a compound having a ring structure configured to contain four or more nitrogen atoms, abrasives, and a liquid medium. The polishing composition can be produced by mixing the polishing removal accelerator containing the compound having the ring structure configured to contain four or more nitrogen atoms, abrasives, and a liquid medium, such as water and an organic solvent.

The polishing composition is suitably used for polishing objects to be polished, such as simple substance silicon, a silicon compounds, and metals, e.g., for polishing the surface containing simple substance silicon, silicon compounds, metals, and the like of a semiconductor wiring board in a process of manufacturing a semiconductor device. The polishing composition is particularly suitably used for polishing silicon nitride. When polishing is performed using the polishing composition, the objects to be polished, such as simple substance silicon, silicon compounds, and metals, particularly silicon nitride, can be polished at a high polishing removal rate.

Hereinafter, the polishing composition of this embodiment is described in detail.
1. Polishing Removal Accelerator

A compound having a ring structure configured to contain four or more nitrogen atoms, i.e., a cyclic compound containing four or more nitrogen atoms in the ring, contributes to an improvement of the polishing removal rate of objects to be polished. In particular, the compound is effective for an improvement of the polishing removal rate of silicon nitride. It is considered that, in the case where the object to be polished is silicon nitride, when the cyclic compound approaches the silicon nitride, the covalent bond of the silicon nitride extends which leads to a reduction in bonding power, and therefore the polishing removal rate is improved.

A heterocyclic compound, such as tetrazole, is added to the polishing composition as an anticorrosive (additive suppressing deterioration (surface roughness and the like) of the surface state of the objects to be polished by suppressing the dissolution of metal of the surface of the objects to be polished) in some cases. However, it is not known that the heterocyclic compound contributes to an improvement of the polishing removal rate of the objects to be polished. More specifically, it is not known that the cyclic compound containing four or more nitrogen atoms in the ring functions as a polishing removal accelerator. It is a matter of course that the cyclic compound containing four or more nitrogen atoms in the ring functions also as the anticorrosive in the polishing composition.

As the cyclic compound containing four or more nitrogen atoms in the ring, tetrazole and a derivative thereof, pentazole and a derivative thereof, and the like are mentioned. Among the above, tetrazole and a derivative thereof are preferable. The number of ring members of the heterocyclic compound is not particularly limited and 5-membered to 14-membered compounds maybe acceptable. As one embodiment, the cyclic compound containing four or more nitrogen atoms in the ring is a five-membered compound or a compound in which two five-membered rings form a single bond. The cyclic compound containing four or more nitrogen atoms in the ring may contain at least one group selected from the group consisting of an amino group, an amide group, a phenyl group, a methyl group, a carboxy group, a phosphate group, a sulfo group, a thiol group (mercapto group), a nitro group, a cyclohexyl group, a hydroxyl group, and the like. Among the above, the cyclic compound preferably contains at least one group selected from the group consisting of an amino group, an amide group, a phenyl group, a carboxy group, a phosphate group, a sulfo group, and a thiol group.

Specific examples of the tetrazole include 1H-tetrazole and 2H-tetrazole.

Specific examples of 1H-tetrazole derivatives include, for example, bistetrazoles, such as 5,5′-bis-1H-tetrazole, 5,5′-bistetrazole diammonium, 5,5′-bistetrazole diaguanidine, and 5,5′-bistetrazole piperazine, 5-amino-1H-tetrazole, 5-phenyl-1H-tetrazole, 5-methyl-1H-tetrazole, 5-carboxy-1H-tetrazole, 5-mercapto-1H-tetrazole, 5-cyclohexyl-1H-tetrazole, 1-amino-1H-tetrazole, 1-phenyl-1H-tetrazole, 1-methyl-1H-tetrazole, 1-carboxy-1H-tetrazole, 1-mercapto-1H-tetrazole, 1-cyclohexyl-1H-tetrazole, 1-amino-5-amino-1H-tetrazole, 1-amino-5-phenyl-1H-tetrazole, 1-amino-5-methyl-1H-tetrazole, 1-amino-5-carboxy-1H-tetrazole, 1-amino-5-mercapto-1H-tetrazole, 1-amino-5-cyclohexyl-1H-tetrazole, 1-phenyl-5-amino-1H-tetrazole, 1-phenyl-5-phenyl-1H-tetrazole, 1-phenyl-5-methyl-1H-tetrazole, 1-phenyl-5-carboxy-1H-tetrazole, 1-phenyl-5-mercapto-1H-tetrazole, 1-phenyl-5-cyclohexyl-1H-tetrazole, 1-methyl-5-amino-1H-tetrazole, 1-methyl-5-phenyl-1H-tetrazole, 1-methyl-5-methyl-1H-tetrazole, 1-methyl-5-carboxy-1H-tetrazole, 1-methyl-5-mercapto-1H-tetrazole, 1-methyl-5-cyclohexyl-1H-tetrazole, 1-carboxy-5-amino-1H-tetrazole, 1-carboxy-5-phenyl-1H-tetrazole, 1-carboxy-5-methyl-1H-tetrazole, 1-carboxy-5-carboxy-1H-tetrazole, 1-carboxy-5-mercapto-1H-tetrazole, 1-carboxy-5-cyclohexyl-1H-tetrazole, 1-mercapto-5-amino-1H-tetrazole, 1-mercapto-5-phenyl-1H-tetrazole, 1-mercapto-5-methyl-1H-tetrazole, 1-mercapto-5-carboxy-1H-tetrazole, 1-mercapto-5-mercapto-1H-tetrazole, 1-mercapto-5-cyclohexyl-1H-tetrazole, 1-cyclohexyl-5-amino-1H-tetrazole, 1-cyclohexyl-5-phenyl-1H-tetrazole, 1-cyclohexyl-5-methyl-1H-tetrazole, 1-cyclohexyl-5-carboxy-1H-tetrazole, 1-cyclohexyl-5-mercapto-1H-tetrazole, 1-cyclohexyl-5-cyclohexyl-1H-tetrazole, N-(1H-tetrazole-5-yl)methacrylamide, 5-(nitroamino)-1H-tetrazole, 5-(hydroxyazo)-1H-tetrazole, 2,5-dihydro-5-thioxo-1H-tetrazole-1-methanesulfonic acid, 5-(nitroamino)-1H-tetrazole, 5-(methyl sulfonate)-1H-tetrazole, 5-(methyl phosphate)-1H-tetrazole, and the like.

Specific examples of 2H-tetrazole derivatives include 5,5′-bis-2H-tetrazole, 5-amino-2H-tetrazole, 5-phenyl-2H-tetrazole, 5-methyl-2H-tetrazole, 5-carboxy-2H-tetrazole, 5-mercapto-2H-tetrazole, 5-cyclohexyl-2H-tetrazole, 2-amino-2H-tetrazole, 2-phenyl-2H-tetrazole, 2-methyl-2H-tetrazole, 2-carboxy-2H-tetrazole, 2-mercapto-2H-tetrazole, 2-cyclohexyl-2H-tetrazole, 2-amino-5-amino-2H-tetrazole, 2-amino-5-phenyl-2H-tetrazole, 2-amino-5-methyl-2H-tetrazole, 2-amino-5-carboxy-2H-tetrazole, 2-amino-5-mercapto-2H-tetrazole, 2-amino-5-cyclohexyl-2H-tetrazole, 2-phenyl-5-amino-2H-tetrazole, 2-phenyl-5-phenyl-2H-tetrazole, 2-phenyl-5-methyl-2H-tetrazole, 2-phenyl-5-carboxy-2H-tetrazole, 2-phenyl-5-mercapto-2H-tetrazole, 2-phenyl-5-cyclohexyl-2H-tetrazole, 2-methyl-5-amino-2H-tetrazole, 2-methyl-5-phenyl-2H-tetrazole, 2-methyl-5-methyl-2H-tetrazole, 2-methyl-5-carboxy-2H-tetrazole, 2-methyl-5-mercapto-2H-tetrazole, 2-methyl-5-cyclohexyl-2H-tetrazole, 2-carboxy-5-amino-2H-tetrazole, 2-carboxy-5-phenyl-2H-tetrazole, 2-carboxy-5-methyl-2H-tetrazole, 2-carboxy-5-carboxy-2H-tetrazole, 2-carboxy-5-mercapto-2H-tetrazole, 2-carboxy-5-cyclohexyl-2H-tetrazole, 2-mercapto-5-amino-2H-tetrazole, 2-mercapto-5-phenyl-2H-tetrazole, 2-mercapto-5-methyl-2H-tetrazole, 2-mercapto-5-carboxy-2H-tetrazole, 2-mercapto-5-mercapto-2H-tetrazole, 2-mercapto-5-cyclohexyl-2H-tetrazole, 2-cyclohexyl-5-amino-2H-tetrazole, 2-cyclohexyl-5-phenyl-2H-tetrazole, 2-cyclohexyl-5-methyl-2H-tetrazole, 2-cyclohexyl-5-carboxy-2H-tetrazole, 2-cyclohexyl-5-mercapto-2H-tetrazole, 2-cyclohexyl-5-cyclohexyl-2H-tetrazole, and the like.

The cyclic compounds containing four or more nitrogen atoms in the ring may be used alone or in combination of two or more types thereof.

Among the heterocyclic compounds, 1H-tetrazole and a derivative thereof are preferable, 1H-tetrazole, 5-amino-1H-tetrazole, 5-phenyl-1H-tetrazole, and bistetrazoles are more preferable, and 1H-tetrazole and 5,5′-bistetrazole diammonium are still more preferable.

The concentration of the cyclic compound containing four or more nitrogen atoms in the ring in the entire polishing composition is preferably 1 mmol/L or more. In such a range, the polishing removal rate of the objects to be polished is improved.

The concentration of the cyclic compound containing four or more nitrogen atoms in the ring in the entire polishing composition is preferably 100 mmol/L or less. In such a range, the cost of the polishing composition can be reduced.
2. Abrasives
2-1 Type

The abrasives have a function of physically polishing the surface of the polishing composition. The type of the abrasives is not particularly limited and oxide particles, such as silica particles, alumina particles, cerium oxide particles, chromium oxide particles, titanium dioxide particles, zirconium dioxide particles, magnesium oxide particles, manganese dioxide particles, zinc oxide particles, and red oxide particles, nitride particles, such as silicon nitride particles and boron nitride particles, carbide particles, such as silicon carbide particles and boron carbide particles, carbonates, such as calcium carbonate and barium carbonate, diamond particles, and the like are mentioned.

Among the specific examples, silica is preferable. Specific examples of silica include silica particles selected from colloidal silica, fumed silica, and sol-gel method silica. Among the silica particles, silica particles selected from colloidal silica and fumed silica, particularly colloidal silica, are preferably used from the viewpoint of reducing scratches generated on the polished surface of the polishing composition. The abrasives may be used alone or in combination of two or more types thereof.
2-2 Aspect Ratio

The aspect ratio of the abrasives, i.e., polishing particles, is preferably less than 1.4, more preferably 1.3 or less, and still more preferably 1.25 or less. Thus, the surface roughness of the objects to be polished caused by the shape of the abrasives can be improved.

The aspect ratio is an average value of values obtained by dividing the length of the long side of a minimum rectangle circumscribing the polishing particles by the length of the short side of the same rectangle and can be determined using general image analysis software from images of the polishing particles obtained by a scanning electron microscope.
2-3 Average Primary Particle Diameter

The average primary particle diameter of the abrasives is preferably 5 nm or more, more preferably 7 nm or more, and still more preferably 10 nm or more. The average primary particle diameter of the abrasives is preferably 200 nm or less, more preferably 150 nm or less, and still more preferably 100 nm or less.

In such a range, the polishing removal rate of the objects to be polished by the polishing composition is improved. Moreover, the occurrence of dishing on the surface of the objects to be polished after performing the polishing using the polishing composition can be further suppressed.

The average primary particle diameter of the abrasives is calculated based on the specific surface area of the abrasives measured by a BET method, for example.
2-4 Average Secondary Particle Diameter

The average secondary particle diameter of the abrasives is preferably 25 nm or more, more preferably 30 nm or more, and still more preferably 35 nm or more. The average secondary particle diameter of the abrasives is preferably 300 nm or less, more preferably 260 nm or less, and still more preferably 220 nm or less.

In such a range, the polishing removal rate of the objects to be polished by the polishing composition is improved. The generation of surface defects on the surface of the objects to be polished after performing the polishing using the polishing composition can be further suppressed.

The secondary particles as used herein refer to particles formed by gathering of the abrasives (primary particles) in the polishing composition. The average secondary particle diameter of the secondary particles can be measured by a dynamic light scattering method, for example.
2-5 Particle Size Distribution

A ratio D90/D10 of a diameter D90 of the particles when the integrated particle mass from the fine particle side in the particle size distribution of the abrasives reaches 90% of the total particle mass to a diameter D10 of the particles when the integrated particle mass from the fine particle side reaches 10% of the total particle mass is preferably 1.5 or more, more preferably 1.8 or more, and still more preferably 2.0 or more. The ratio D90/D10 is preferably 5.0 or less and more preferably 3.0 or less.

In such a range, the polishing removal rate of the objects to be polished is improved and the generation of surface defects on the surface of the objects to be polished after performing the polishing using the polishing composition can be further suppressed.

The particle size distribution of the abrasives can be determined by a laser diffraction scattering method, for example.
2-6 Content of Abrasives

The content of the abrasives in the entire polishing composition is preferably 0.005% by mass or more, more preferably 0.5% by mass or more, still more preferably 1% by mass or more, and particularly preferably 2% by mass or more. In such a range, the polishing removal rate of the objects to be polished is improved.

The content of the abrasives in the entire polishing composition is preferably 50% by mass or less, more preferably 30% by mass or less, and still more preferably 20% by mass or less. In such a range, the cost of the polishing composition can be reduced. Moreover, the generation of surface defects on the surface of the objects to be polished after performing the polishing using the polishing composition can be further suppressed.
2-7 Surface Modification

The abrasives may be surface modified abrasives. The surface modified abrasives can be obtained by mixing metals, such as aluminum, titanium, and zirconium, or oxides thereof with abrasives not subjected to surface modification, and then doping the surface of the abrasives with the metals, such as aluminum, titanium, and a zirconium, or the oxides thereof or by immobilizing organic acid on the surface of the abrasives, for example. Among the surface modified polishing particles, colloidal silica in which organic acid is immobilized is particularly preferable.

The immobilization of the organic acid on the surface of the colloidal silica is performed by chemically bonding functional groups of the organic acid on the surface of the colloidal silica, for example. By merely causing the colloidal silica and the organic acid to coexist, the immobilization of the organic acid on the colloidal silica is not achieved.

When sulfonic acid which is one type of the organic acid is immobilized on colloidal silica, the immobilization can be performed by the method described in “Sulfonic acid-functionalized silica through quantitative oxidation of thiol groups”, Chem. Commun. 246-247 (2003), for example.

Specifically, a silane coupling agent having a thiol group, such as 3-mercaptopropyl trimethoxysilane, is caused to react to a hydroxy group of the surface of the colloidal silica for coupling, and then the thiol group is oxidized with hydrogen peroxide, whereby the colloidal silica on the surface of which sulfonic acid is immobilized can be obtained.

Or, when carboxylic acid is immobilized on the surface of colloidal silica, the immobilization can be performed by the method described in “Novel Silane Coupling Agents Containing a Photolabile 2-Nitrobenzyl Ester for Introduction of a Carboxy Group on the Surface of Silica Gel”, Chemistry Letters, 3, 228-229 (2000), for example. Specifically, a silane coupling agent containing photolabile 2-nitrobenzyl ester is caused to react to a hydroxy group of the surface of the colloidal silica for coupling, and then light is emitted, whereby the colloidal silica on the surface of which carboxylic acid is immobilized can be obtained.

Alternatively, organic acids, such as sulfinic acid and phosphonic acid, may be immobilized on the surface of colloidal silica.

In usual colloidal silica, a zeta potential value is close to zero under acidic conditions, and therefore, colloidal silica particles are likely to cause aggregation without electrically repelling each other under the acidic conditions. On the other hand, the colloidal silica on the surface of which the organic acid is immobilized is surface modified in such a manner that the zeta potential value has a relatively large negative value also under the acidic conditions, and therefore colloidal silica particles strongly repel each other and are satisfactorily dispersed also under the acidic conditions. As a result, the storage stability of the polishing composition is improved.
3. Liquid Medium

The liquid medium may be liquid capable of dispersing or dissolving the components (a cyclic compound containing four or more nitrogen atoms in the ring, abrasives, additives, and the like) of the polishing composition and is not particularly limited when the liquid functions as a dispersion medium or a solvent. As the liquid medium, water and organic solvents are mentioned and can be used alone or as a mixture of two or more types thereof. The liquid medium preferably contains water. However, it is preferable to use water free from impurities as much as possible from the viewpoint of preventing blocking of an action of each component. Specifically, pure water or ultrapure water from which impurity ions are removed with an ion exchange resin, and then foreign substances are removed through a filter or distilled water is preferable.
4. Additive

To the polishing composition, various additives, such as a pH adjuster, an oxidizer, a complexing agent, a surfactant, a water-soluble polymer, and an antifungal agent, may be added for increasing the performance thereof. 4-1 PH adjuster

The pH value of the polishing composition is preferably 1.5 or more and more preferably 2 or more. When the pH value of the polishing composition is higher, the dissolution of the objects to be polished is more likely to occur, and therefore the polishing removal rate of the objects to be polished by the polishing composition is improved in such a range. Moreover, with a reduction in the pH value of the polishing composition, the handling thereof is further facilitated. Therefore, the pH value of the polishing composition is preferably less than 12 and more preferably 10 or less.

The pH value of the polishing composition can be adjusted by the addition of the pH adjuster. The pH adjuster to be used as necessary for adjusting the pH value of the polishing composition to a desired value may be either acid or alkali and may be either an inorganic compound or an organic compound.

Specific examples of the acid as the pH adjuster include inorganic acids or organic acids, such as carboxylic acid and organic sulfuric acid. Specific examples of the inorganic acids include sulfuric acid, nitric acid, boric acid, carbonic acid, hypophosphorous acid, phosphorous acid, phosphoric acid, and the like. Specific examples of the carboxylic acid include formic acid, acetic acid, propionic acid, butyric acid, valeric acid, 2-methylbutyric acid, n-hexanoic acid, 3,3-dimethyl butyric acid, 2-ethyl butyric acid, 4-methylpentanoic acid, n-heptanoic acid, 2-methyl hexanoic acid, n-octanoic acid, 2-ethyl hexanoic acid, benzoic acid, glycolic acid, salicylic acid, glyceric acid, oxalic acid, malonic acid, succinic acid, glutaric acid, adipic acid, pimelic acid, maleic acid, phthalic acid, malic acid, tartaric acid, citric acid, lactic acid, and the like.

Specific examples of the organic sulfuric acid include methanesulfonic acid, ethane sulfonic acid, isethionic acid, and the like. The acids may be used alone or in combination of two or more types thereof.

Specific examples of bases as the pH adjuster include hydroxides of alkali metals or salts thereof, hydroxides of alkaline earth metals or salts thereof, quaternary ammonium hydroxides or salts thereof, ammonia, amine, and the like.

Specific examples of the alkali metals include potassium, sodium, and the like. Specific examples of the alkaline earth metals include calcium, strontium, and the like. Specific examples of the salts include carbonates, hydrogencarbonates, sulfates, acetates, and the like. Specific examples of the quaternary ammonium include tetramethylammonium, tetraethylammonium, tetrabutyl ammonium, and the like.

As quaternary ammonium hydroxide compounds, quaternary ammonium hydroxides or salts thereof are included. Specific examples thereof include tetramethylammonium hydroxide, tetraethylammonium hydroxide, tetrabutylamrnonium hydroxide, and the like are mentioned.

Specific examples of the amine include methylamine, dimethylamine, trimethylamine, ethylamine, diethylamine, triethylamine, ethylene diamine, monoethanolamine, N-(β-aminoethyl)ethanolamine, hexamethylenediamine, diethylenetriamine, triethylenetetramine, anhydrous piperazine, piperazine hexahydrate, 1-(2-aminoethyl)piperazine, N-methyl piperazine, guanidine, and the like.

The bases may be used alone or in combination of two or more types thereof.

Among the bases, ammonia, ammonium salts, alkali metal hydroxides, alkali metal salts, quaternary ammonium hydroxide compounds, and amines are preferable and ammonia, potassium compounds, sodium hydroxides, quaternary ammonium hydroxide compounds, ammonium hydrogencarbonates, ammonium carbonates, sodium hydrogencarbonates, and sodium carbonates are more preferable.

The polishing composition more preferably further contains potassium compounds as the base from the viewpoint of preventing metal pollution. As the potassium compounds, potassium hydroxides or potassium salts are mentioned and, specifically, potassium hydroxides, potassium carbonates, potassium hydrogencarbonates, potassium sulfates, potassium acetates, potassium chlorides, and the like are mentioned.
4-2 Oxidizer

To the polishing composition, oxidizers may be added for oxidizing metal to form an oxide film to facilitate polishing. Specific examples of the oxidizers include hydrogen peroxides, peracetic acids, percarbonates, urea peroxides, perchloric acids, persulfates, and the like. Specific examples of the persulfates include sodium persulfate, potassium persulfate, ammonium persulfate, and the like. The oxidizers may be used alone or in combination of two or more types thereof. Among the oxidizers, persulfates and hydrogen peroxides are preferable and hydrogen peroxides are particularly preferable.

When the content of the oxidizer in the entire polishing composition is higher, the polishing removal rate of objects to be polished by the polishing composition is further improved. Therefore, the content of the oxidizer in the entire polishing composition is preferably 0.01% by mass or more, more preferably 0.05% by mass or more, and still more preferably 0.1% by mass or more.

When the content of the oxidizer in the entire polishing composition is lower, the material cost of the polishing composition can be further reduced. A load of the treatment of the polishing composition after used for polishing, i.e., waste liquid treatment, can be reduced. Furthermore, excessive oxidation of the surface of objects to be polished by the oxidizer becomes unlikely to occur. Therefore, the content of the oxidizer in the entire polishing composition is preferably 10% by mass or less, more preferably 5% by mass or less, and still more preferably 3% by mass or less.
4-3 Complexing Agent

To increase the polishing removal rate of objects to be polished by the polishing composition, a complexing agent may be added to the polishing composition. The complexing agent has an action of chemically etching the surface of objects to be polished. Specific examples of the complexing agent include inorganic acids or salts thereof, organic acids or salts thereof, nitrile compounds, amino acids, chelating agents, and the like. The complexing agents may be used alone or in combination of two or more types thereof. For the complexing agents, commercially-available items may be used or synthetic compounds may be used.

Specific examples of the inorganic acids include hydrochloric acid, sulfuric acid, nitric acid, carbonic acid, boric acid, tetrafluoroboric acid, hypophosphorous acid, phosphorous acid, phosphoric acid, pyrophoric acid, and the like.

Specific examples of the organic acids include carboxylic acids, sulfonic acids, and the like. Specific examples of the carboxylic acids include monovalent carboxylic acids, such as formic acid, acetic acid, propionic acid, butyric acid, valeric acid, 2-methylbutyric acid, n-hexanoic acid, 3,3-dimethyl butyric acid, 2-ethyl butyric acid, 4-methylpentanoic acid, n-heptanoic acid, 2-methyl hexanoic acid, n-octanoic acid, 2-ethyl hexanoic acid, lactic acid, glycolic acid, glyceric acid, benzoic acid, and salicylic acid, and polyvalent carboxylic acids, such as oxalic acid, malonic acid, succinic acid, glutaric acid, gluconic acid, adipic acid, pimelic acid, maleic acid, phthalic acid, fumaric acid, malic acid, tartaric acid, and citric acid. Specific examples of the sulfonic acids include methanesulfonic acid, ethane sulfonic acid, isethionic acid, and the like.

As the complexing agent, salts of the inorganic acids or the organic acids are usable. Particularly when salts of weak acids and strong bases, salts of strong acids and weak bases, or salts of weak acids and weak bases are used, a pH buffering action can be expected. Specific examples of such salts include potassium chloride, sodium sulfate, potassium nitrate, potassium carbonate, potassium tetrafluoroborate, potassium pyrophosphate, potassium oxalate, trisodium citrate, (+)− potassium tartrate, potassium hexafluorophosphate, and the like.

Specific examples of the nitrile compounds include acetonitrile, aminoacetonitrile, propionitrile, butyronitrile, isobutyronitrile, benzonitrile, glutarodinitrile, methoxyacetonitrile, and the like.

Specific examples of the amino acids include glycine, α-alanine, β-alanine, N-methyl glycine, N,N-dimethyl glycine, 2-aminobutyric acid, norvaline, valine, leucine, norleucine, isoleucine, phenylalanine, proline, sarcosine, ornithine, lysine, taurine, serine, threonine, homoserine, tyrosine, bicin, tricine, 3,5-diiodotyrosine, β-(3,4-dihydroxyphenyl)alanine, thyroxine, 4-hydroxyproline, cystein, methionine, ethionine, lanthionine, cystathionine, cystine, cysteic acid, aspartic acid, glutamic acid, S-(carboxymethyl)cystein, 4-aminobutyric acid, asparagine, glutamine, azaserine, arginine, canavanine, citrulline, δ-hydroxylysine, creatine, histidine, 1-methyl histidine, 3-methyl histidine, and tryptophan.

Specific examples of the chelating agents include nitrilotriacetic acid, diethylenetriamine pentaacetic acid, ethylenediaminetetraacetic acid, N,N,N-trimethylene phosphonic acid, ethylene diamine-N,N,N′,N′-tetramethylene sulfonic acid, trans-cyclohexane diamine tetraacetic acid, 1,2-diaminopropane tetraacetic acid, glycol ether diamine tetraacetic acid, ethylene diamine olthohydroxy phenylacetic acid, ethylene diamine disuccinic acid (SS isomer), N-(2-carboxylate ethyl)-L-aspartic acid, β-alanine diacetic acid, 2-phosphonobutane-1,2,4-tricarboxylic acid, 1-hydroxy ethylidene-1,1-diphosphonic acid, N,N′-bis(2-hydroxybenzyl)ethylene diamine-N,N′diacetic acid, 1,2-dihydroxy benzene-4,6-disulfonic acid, and the like.

Among the above, at least one type selected from the group consisting of inorganic acids or salts thereof, carboxylic acids or salts thereof, and nitrile compounds is preferable. From the viewpoint of the stability of the complex structure with a metallic compound contained in objects to be polished, inorganic acids or salts thereof are more preferable. When those having a ph adjustment function (for example, various types of acids) are used as the various types of complexing agents mentioned above, the complexing agents may be used as at least one part of the pH adjuster.

The lower limit of the content of the complexing agent in the entire polishing composition is not particularly limited because a small amount of the complexing agent exhibits effects. However, the polishing removal rate of the objects to be polished by the polishing composition is improved when the content of the complexing agent is higher, and therefore the content of the complexing agent in the entire polishing composition is preferably 0.001 g/L or more, more preferably 0.01 g/L or more, and still more preferably 1 g/L or more.

When the content of the complexing agent in the entire polishing composition is lower, the dissolution of objects to be polished is more unlikely to occur and the level difference elimination properties are improved. Therefore, the content of the complexing agent in the entire polishing composition is preferably 20 g/L or less, more preferably 15 g/L or less, and still more preferably 10 g/L or less.
4-4 Surfactant

To the polishing composition, surfactants maybe added. The surfactants have an action of giving hydrophilicity to the polished surface of objects to be polished after polishing, and therefore can improve the cleaning efficiency of the objects to be polished after polishing and can prevent the adhesion of dirt and the like. As the surfactants, anionic surfactants, cationic surfactants, amphoteric surfactants, and nonionic surfactants are all usable.

Specific examples of the anionic surfactants include polyoxyethylene alkyl ether acetate, polyoxyethylene alkyl sulfate ester, alkyl sulfate ester, polyoxyethylene alkyl sulfate, alkyl sulfate, alkyl benzene sulfonate, alkyl phosphate ester, polyoxyethylene alkyl phosphate ester, polyoxyethylene sulfosuccinate, alkyl sulfosuccinate, alkyl naphthalene sulfonate, alkyl diphenyl ether disulfonate, or salts thereof.

Specific examples of the cationic surfactants include alkyltrimethylammonium salts, alkyldimethylammonium salts, alkylbenzyldimethylammonium salts, and alkyl amine salts.

Specific examples of the amphoteric surfactants include alkyl betaine and alkylamine oxide.

Specific examples of the nonionic surfactants include polyoxyethylene alkylether, polyoxyalkylene alkylether, sorbitan fatty acid ester, glycerol fatty acid ester, polyoxyethylene fatty acid ester, polyoxyethylene alkylamine, and alkylalkanolamide.

The surfactants may be used alone or in combination of two or more types thereof.

When the content of the surfactant in the entire polishing composition is higher, the cleaning efficiency of objects to be polished after polishing is further improved. Therefore, the content of the surfactant in the entire polishing composition is preferably 0.0001 g/L or more and more preferably 0.001 g/L or more.

When the content of the surfactant in the entire polishing composition is lower, the residual amount of the surfactant to the polished surface of objects to be polished after polishing is further reduced and the cleaning efficiency is further improved. Therefore, the content of the surfactant in the entire polishing composition is preferably 10 g/L or less and more preferably 1 g/L or less.
4-5 Water-Soluble Polymer

To the polishing composition, a water-soluble polymer maybe added. When the water-soluble polymer is added to the polishing composition, the surface roughness of objects to be polished after polishing further decreases (becomes smooth).

Specific examples of the water-soluble polymer include polystyrene sulfonate, polyisoprene sulfonate, polyacrylate, polymaleic acid, polyitaconic acid, polyvinyl acetate, polyvinyl alcohol, polyglycerol, polyvinyl pyrrolidone, a copolymer of isoprene sulfonic acid and acrylic acid, a polyvinyl pyrrolidone polyacrylate copolymer, a polyvinyl pyrrolidone vinyl acetate copolymer, a salt of a naphthalene sulfonic acid formalin condensate, a diallylamine hydrochloride sulfur dioxide copolymer, carboxymethylcellulose, a salt of carboxymethylcellulose, hydroxyethylcellulose, hydroxypropylcellulose, pullulan, chitosan, and chitosan salts. The water-soluble polymers may be used alone or in combination of two or more types thereof.

When the content of the water-soluble polymer in the entire polishing composition is higher, the surface roughness of the polished surface of objects to be polished further decreases. Therefore, the content of the water-soluble polymer in the entire polishing composition is preferably 0.0001 g/L or more and more preferably 0.001 g/L or more.

When the content of the water-soluble polymer in the entire polishing composition is lower, the residual amount of the water-soluble polymer to the polished surface of objects to be polished is further reduced and the cleaning efficiency is further improved. Therefore, the content of the water-soluble polymer in the entire polishing composition is preferably 10 g/L or less and more preferably 1 g/L or less.
4-6 Antifungal Agent and Antiseptic

To the polishing composition, antifungal agents and antiseptics may be added. Specific examples of the antifungal agents and the antiseptics include isothiazoline-based antiseptics (for example, 2-methyl-4-isothiazoline-3-one and 5-chloro-2-methyl-4-isothiazoline-3-one) p-hydroxybenzoate esters, and phenoxyethanol. The antifungal agents and the antiseptics may be used alone or in combination of two or more types thereof.
5. Method for Producing Polishing Composition

A method for producing a polishing composition of this embodiment is not particularly limited and the polishing composition can be produced by stirring and mixing a cyclic compound containing four or more nitrogen atoms in the ring, abrasives, and, as desired, various types of additives in a liquid medium, such as water.

The temperature in the mixing is not particularly limited and is preferably 10° C. or more and 40° C. or less. Heating may be performed for increasing the dissolution rate. Moreover, the mixing time is also not particularly limited.
6. Objects to be Polished

The type of the objects to be polished is not particularly limited and simple substance silicon, a silicon compound, metal, and the like are mentioned as one embodiment. The simple substance silicon and the silicon compound are objects to be polished having a layer containing a silicon containing material.

As the single silicon include monocrystalline silicon, polycrystalline silicon (polysilicon), amorphous silicon, and the like are mentioned, for example. As the silicon compound, silicon nitride, silica dioxide, silicon carbide, and the like are mentioned, for example. A silicon compound film contains a low dielectric constant film having a relative dielectric constant of 3 or less.

As the metal, tungsten, copper, aluminum, hafnium, cobalt, nickel, titanium, tantalum, gold, silver, platinum, palladium, rhodium, ruthenium, iridium, osmium, and the like are mentioned, for example. The metals may be contained in the form of an alloy or a metallic compound. Among the metals, copper is preferable.

The objects to be polished may be substrates having a positively charged region when contacting the polishing composition in a state where the ph of the polishing composition is 6 or less.
7. Polishing Method

The configuration of a polishing device is not particularly limited and, for example, general polishing devices are usable which have a holder holding a substrate and the like having objects to be polished, an actuator, such as a motor, capable of changing the rotational speed, and a polishing platen to which a polishing pad (polishing cloth) can be stuck. As the polishing pad, general nonwoven fabrics, polyurethane, porous fluororesin, and the like can be used without being particularly limited. For the polishing pad, one subjected to groove processing so as to collect a liquid polishing composition is usable.

The polishing conditions are not particularly limited and, for example, the rotational speed of the polishing platen can be set to 10 min⁻¹ or more and 500 min⁻¹ or less. The pressure (polishing pressure) applied to the substrate having objects to be polished can be set to 0.7 kPa or more and 69 kPa or less.

A method for supplying the polishing composition to the polishing pad is also not particularly limited and, for example, a method for continuously supplying the polishing composition with a pump or the like is adopted. The supply amount of the polishing composition is not limited. It is preferable that the surface of the polishing pad is always covered with the polishing composition. In the polishing of objects to be polished, the polishing may be performed using an undiluted solution of the polishing composition of this embodiment as it is and the polishing may be performed using an undiluted solution of the polishing composition diluted to 10 or more fold with a diluted solution, such as water.

After the polishing, the substrate is cleaned with running water, for example, water droplets adhering onto the substrate are shaken off with a spin drier or the like, and then the substrate is dried, whereby the substrate having a layer containing a silicon containing material is obtained, for example.

Thus, the polishing composition of this embodiment is usable for polishing of a substrate. More specifically, the surface of the substrate can be polished at a high polishing removal rate by a method including polishing the surface of a substrate using the polishing composition of this embodiment, and thus the substrate can be produced. As the substrate, a silicon wafer having a layer containing simple substance silicon, a silicon compound, metal, and the like is mentioned, for example.

### EXAMPLES

Hereinafter, the present invention is more specifically described with reference to Examples but the present invention is not particularly limited to the Examples.

### <Preparation of Polishing Composition>

### Example 1

To water as a dispersion medium (liquid medium), by mass of colloidal silica (Average primary particle diameter: 32 nm, Average secondary particle diameter: 70 nm) on the surface of which sulfonic acid is immobilized was added and 1H-tetrazole was added in such a manner that the concentration was 10 mmol/L based on the entire composition, and then stirred and mixed to give a polishing composition (Mixing temperature of about 25° C., Mixing time: about 10 minutes). Herein, the pH of the polishing composition was adjusted with a pH adjuster, maleic acid or potassium hydroxide (KOH), in such a manner that the pH was 4.0. The pH was measured with a pH meter (manufactured by HORIBA, LTD., Model: LAQUA).

### Examples 2 to 4 and Comparative Examples 1 to 3

Polishing compositions were prepared by the same operation as that of Example 1, except using various types of nitrogen containing heterocyclic compounds shown in the following table 1.

Table 1 also shows the size and the concentration of colloidal silica, the pH of each polishing composition, and the number of nitrogen atoms in the nitrogen containing heterocyclic compound contained in each polishing removal accelerator and the type of functional groups added to the nitrogen containing heterocyclic compound used in Examples and Comparative Examples.

### <Characteristic Evaluation of Polishing Compositions>

The characteristics of the polishing compositions prepared by the above-described operation were evaluated as follows.

### (Polishing Removal Rate of Silicon Nitride)

The polishing removal rate was evaluated by determining the film thicknesses before and after the polishing with an optical interference type film thickness meter for a 200 mm wafer on which a silicon nitride (SiN) film was formed, and then dividing a difference between the film thicknesses before and after the polishing by polishing time. Herein, the polishing conditions are as follows.

### [Polishing Conditions]

Polishing machine: CMP one side polishing machine for 200 mm wafers
Pad: Polyurethane pad
Pressure: 3.2 psi (about 22.1 kPa)
Number of rotations of platen: 90 rpm
Flow rate of polishing composition: 130 ml/min
Polishing time: For 1 minute
Objects to be polished: Wafer with SiN film (200 mm)

SiN film formation method: Low pressure chemical vapor deposition method (LPCVD)

Film thickness of SiN film: 3500 Å

The polishing removal rate measurement results are also shown in Table 1.

TABLE 1

| | Colloidal silica | | | Polishing removal accelerator | | | | |
|---|---|---|---|---|---|---|---|---|
| | Size | | | | | Number of | Additional | Polishing removal |
| | (Average secondary | | | Nitrogen containing | Concentration | nitrogen atoms (N) in | functional | rate of SiN |
| | particle diameter) | Concentration | pH | heterocyclic compound | [mmol/L] | heterocyclic ring | group | [Å/min] |
| Comp. | 70 nm | 2% | 4 | None | 0 | 0 | None | 296 |
| Ex. 1 | | | | | | | | |
| Comp. | 70 nm | 2% | 4 | Imidazole | 10 | 2 | None | 358 |
| Ex. 2 | | | | | | | | |
| Comp. | 70 nm | 2% | 4 | 1,2,4-triazole | 10 | 3 | None | 367 |
| Ex. 3 | | | | | | | | |
| Ex. 1 | 70 nm | 2% | 4 | 1H-tetrazole | 10 | 4 | None | 399 |
| Ex. 2 | 70 nm | 2% | 4 | 5-amino-1H-tetrazole | 10 | 4 | Amino group | 379 |
| Ex. 3 | 70 nm | 2% | 4 | 5-phenyl-1H-tetrazole | 10 | 4 | Phenyl group | 379 |
| Ex. 4 | 70 nm | 2% | 4 | 5,5′-bistetrazole | 10 | 4 | None (bi-) | 462 |
| | | | | diammonium | | | | |

The results shown in Table 1 show that, when silicon nitride is polished using the polishing compositions of Examples 1 to 4, all wafers can be polished at a higher polishing removal rate than that when the polishing compositions of Comparative Examples 1 to 3 were used. In particular, the results of the polishing compositions of Comparative Examples 2 and 3 show that the polishing removal rates in the polishing compositions containing cyclic compounds containing 3 or less nitrogen atoms in the ring are inferior to Examples 1 to 4 and the use of a cyclic compound containing four or more nitrogen atoms in the ring is important.

Moreover, it is found that the polishing removal rate of silicon nitride of the polishing compositions of Examples 1 and 4 is higher than that of the polishing compositions of Examples 2 and 3. The results can be construed as follows.

### (Coverage by Nitrogen Containing Heterocyclic Group)

As shown in Table 1, the nitrogen containing heterocyclic compounds contained in the polishing compositions of Examples 1 and 4 each are free from an additional functional group. On the other hand, the nitrogen containing heterocyclic compounds contained in the polishing compositions of Examples 2 and 3 each have an additional functional group.

Herein, when it is supposed that the entire surface of objects to be polished is covered with each of the nitrogen containing heterocyclic compounds used in Examples 1 to 4, the coating amount (coverage) of the surface of the objects to be polished by the additional functional groups is a non-negligible amount (value) (about 30 to 50%). As a result, the coating amount (coverage) of the surface of the objects to be polished by the nitrogen containing heterocyclic group (tetrazole ring) is larger in the nitrogen containing heterocyclic compounds free from the additional functional groups than in the nitrogen containing heterocyclic compounds containing the additional functional groups.

The polishing removal rate generally tends to be dependent on the coverage of the surface of the objects to be polished surface. Therefore, it is considered that the polishing removal rate of silicon nitride was higher in the polishing compositions of Examples 1 and 4 in which the coverage of the surface of the objects to be polished is relatively high than in the polishing compositions of Examples 2 and 3 in which the coverage of the surface of the objects to be polished is relatively low.

(Distance between Nitrogen Containing Heterocyclic Compound and Surface of Objects to be Polished)

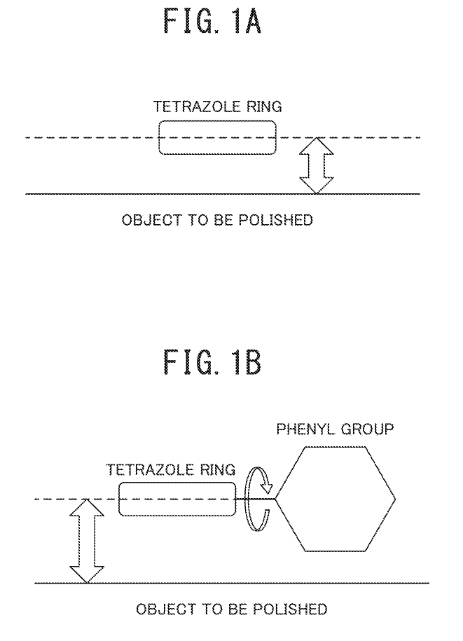

FIG. 1A is a side view schematically illustrating a state where 1H-tetrazole contained in the polishing composition of Example 1 approached an object to be polished. FIG. 1B is a side view schematically illustrating a state where 5-phenyl-1H-tetrazole contained in the polishing composition of Example 3 approached an object to be polished.

As described above, it is considered that, in the case where the object to be polished is silicon nitride, when the nitrogen containing heterocyclic compound approaches the silicon nitride, the covalent bond of the silicon nitride extends which leads to a reduction in bonding power, and thus the polishing removal rate is improved.

When the nitrogen containing heterocyclic compound is 1H-tetrazole, the 1H-tetrazole is free from an additional functional group, and therefore the nitrogen containing heterocyclic ring can sufficiently approach within a distance where the nitrogen containing heterocyclic ring can interact with the surface atoms of the object to be polished.

On the other hand, when the nitrogen containing heterocyclic compound is 5-phenyl-1H-tetrazole, the 5-phenyl-1H-tetrazole has an additional functional group, and therefore the nitrogen containing heterocyclic ring cannot sufficiently approach within a distance where the nitrogen containing heterocyclic ring can interact with the surface atoms of the object to be polished. This is because the phenyl group and the tetrazole ring form a single bond, and therefore the phenyl group freely rotates to be steric hindrance at a temperature (normal temperature) where the polishing composition is used.

As a result, it is considered that the polishing removal rate of silicon nitride is higher in the IH-tetrazole, the nitrogen containing heterocyclic ring of which can further approach the surface atoms of the object to be polished, than in the 5-phenyl-1H-tetrazole.

The above-described construction is applicable also to 5-amino-1H-tetrazole contained in the polishing composition of Example 2 and 5,5′-bistetrazole diammonium contained in the polishing composition of Example 5.

What is claimed is:

1. A polishing composition comprising:
a polishing removal accelerator containing a compound having a ring structure configured to contain four or more nitrogen atoms;
abrasives; and
a liquid medium.

2. The polishing composition according to claim 1, wherein
a number of ring members of the ring structure is 5 or more and 14 or less.

3. The polishing composition according to claim 1, wherein
the compound having the ring structure contains at least one group selected from the group consisting of an amino group, an amide group, a phenyl group, a carboxy group, a phosphate group, a sulfo group, and a thiol group.

4. The polishing composition according to claim 1, wherein
the compound having the ring structure is at least one type selected from the group consisting of tetrazole and a derivative of the tetrazole.

5. The polishing composition according to claim 4, wherein
the tetrazole and the derivative of the tetrazole include at least one type selected from the group consisting of 1H-tetrazole, 5-amino-1H-tetrazole, 5-phenyl-1H-tetrazole, and 5,5-bistetrazole diammonium.

6. The polishing composition according to claim 1, wherein
a concentration of the compound having the ring structure is 1 mmol/L or more and 100 mmol/L or less.

7. The polishing composition according to claim 1, wherein
the polishing composition is used for polishing of a substrate having a positively charged region when contacting the polishing composition in a state where pH is 6 or less.

8. A method for producing the polishing composition according to claim 1, the method comprising:
mixing the polishing removal accelerator, the abrasives, and the liquid medium.

9. A polishing method comprising:
polishing an object to he polished using the polishing composition according to claim 1.

10. A method for producing a substrate:
comprising polishing a surface of a substrate using the polishing composition according to claim 1.

11. The polishing composition according to claim 2, wherein
the compound having the ring structure contains at least one group selected from the group consisting of an amino group, an amide group, a phenyl group, a carboxy group, a phosphate group, a sulfo group, and a thiol group.

12. The polishing composition according to claim 2, wherein
the compound having the ring structure is at least one type selected from the group consisting of tetrazole and a derivative of the tetrazole.

13. The polishing composition according to claim 3, wherein
the compound having the ring structure is at least one type selected from the group consisting of tetrazole and a derivative of the tetrazole.

14. The polishing composition according to claim 2, wherein
a concentration of the compound having the ring structure is 1 mmol/L or more and 100 mmol/L or less.

15. The polishing composition according to claim 3, wherein
a concentration of the compound having the ring structure is 1 mmol/L or more and 100 mmol/L or less.

16. The polishing composition according to claim 4, wherein
a concentration of the compound having the ring structure is 1 mmol/L or more and 100 mmol/L or less.

17. The polishing composition according to claim 5, wherein
a concentration of the compound having the ring structure is 1 mmol/L or more and 100 mmol/L or less.

18. The polishing composition according to claim 2, wherein
the polishing composition is used for polishing of a substrate having a positively charged region in a state where pH of the polishing composition is 6 or less.

19. The polishing composition according to claim 3, wherein
the polishing composition is used for polishing of a substrate having a positively charged region in a state where pH of the polishing composition is 6 or less.

20. The polishing composition according to claim 4, wherein
the polishing composition is used for polishing of a substrate having a positively charged region in a state where pH of the polishing composition is 6 or less.
